(19) United States
(12) Patent Application Publication
(10) Pub. No.: US 2022/0355107 A1
(43) Pub. Date: Nov. 10, 2022

(54) APPARATUS AND METHOD FOR AUTOMATED CONTROL OF A TRANSCUTANEOUS ELECTRICAL NERVE STIMULATION (TENS) DEVICE BASED ON TENS USER'S ACTIVITY TYPE, LEVEL AND DURATION

(71) Applicant: Neurometrix, Inc., Woburn, MA (US)

(72) Inventors: Shai N. Gozani, Newton, MA (US); Xuan Kong, Acton, MA (US); Martin J. Moynihan, Waltham, MA (US)

(21) Appl. No.: 17/313,441

(22) Filed: May 6, 2021

(51) Int. Cl. A61N 1/36

(52) U.S. Cl. CPC A61N 1/36031; A61N 1/36034; A61N 1/0456

(57) ABSTRACT

Apparatus for providing transcutaneous electrical nerve stimulation (TENS) therapy to a user, said apparatus comprising: a stimulation unit for electrically stimulating at least one nerve of the user; a sensing unit for sensing body movement of the user to analyze body movement activity type and activity duration; an application unit for providing mechanical coupling between said sensing unit and the user's body; and a feedback unit for at least one of (i) providing the user with feedback in response to said analysis of said body movement activity type and activity duration of the user, and (ii) modifying the electrical stimulation provided to the user by said stimulation unit in response to said analysis of said body movement activity type and activity duration of the user.

### FIELD OF THE INVENTION

This invention relates generally to Transcutaneous Electrical Nerve Stimulation (TENS) devices that deliver electrical currents across the intact skin of a user to provide symptomatic relief of pain. More specifically, this invention relates to apparatus and methods for analyzing TENS user's activity type, level, and duration based on motion-tracking sensor data such as that provided by an accelerometer incorporated within the TENS device. Data from other sensors are considered as well, including electromyograph sensors, acoustic myograph sensors, force sensors, and stretchable conductive sensors. Operations of the TENS device are modified based on the user's activity type, level, and duration.

### BACKGROUND OF THE INVENTION

Transcutaneous electrical nerve stimulation (TENS) is the delivery of electricity (i.e., electrical stimulation) across the intact surface of a user's skin in order to activate sensory nerve fibers. The most common application of TENS therapy is to provide analgesia, such as for alleviation of chronic pain. Other applications of TENS therapy include, but are not limited to, reducing the symptoms of restless leg syndrome, decreasing nocturnal muscle cramps, and providing relief from generalized pruritus.

Movement-evoked pain is the pain that worsens when a person is engaged in physical activities such as exercising and walking. Physical activity is recognized as an important part of disease management, such as that for fibromyalgia management. However, patients often report activity-dependent deep tissue pains that prevent them from receiving the full benefit by completing prescribed exercise regiments.

Movement-evoked pain is believed to be associated with hyperalgesia and central sensitization. Pressure pain threshold (PPT) is an experimental measure of deep tissue pain sensitivity. Low PPT is associated with high sensitivity to movement-evoked musculoskeletal pain. A newly-developed wearable TENS device (i.e., the Quell® device, Neurometrix, Inc., Woburn, Mass., USA) is found to increase PPT in fibromyalgia patients. Therefore, TENS therapies from devices like the Quell® device are expected to reduce movement-related pain.

A conceptual model for how sensory nerve stimulation leads to pain relief was proposed by Melzack and Wall in 1965. Their theory proposes that the activation of sensory nerves (Aβ fibers) closes a “pain gate” in the spinal cord that inhibits the transmission of pain signals carried by nociceptive afferents (C and Aδ fibers) to the brain. In the past 20 years, anatomic pathways and molecular mechanisms that may underlie the pain gate have been identified. Sensory nerve stimulation (e.g., via TENS) activates the descending pain inhibition system, primarily the periaqueductal gray (PAG) and rostroventral medial medulla (RVM) located in the midbrain and medulla sections of the brainstem, respectively. The PAG has neural projections to the RVM, which in turn has diffuse bilateral projections into the spinal cord dorsal horn that inhibit ascending pain signal transmission.

TENS is typically delivered in short discrete pulses, with each pulse typically being several hundred microseconds in duration, at frequencies between about 10 and 150 Hz, through hydrogel electrodes placed on the user's body. TENS is characterized by a number of electrical parameters including the amplitude and shape of the stimulation pulse (which combine to establish the pulse charge), the frequency and pattern of the pulses, the duration of a therapy session, and the interval between therapy sessions. All of these parameters are correlated to the therapeutic dose. For example, higher amplitude and longer pulses (i.e., larger pulse charge) increase the dose, whereas shorter therapy sessions decrease the dose. Clinical studies suggest that pulse charge and therapy session duration have the greatest impact on therapeutic dose.

To achieve maximum pain relief (i.e., hypoalgesia), TENS needs to be delivered at an adequate stimulation intensity. Intensities below the threshold of sensation are not clinically effective. The optimal therapeutic intensity is often described as one that is “strong yet comfortable”. Most TENS devices rely on the user to set the stimulation intensity, usually through a manual intensity control comprising an analog intensity knob or digital intensity control push-buttons. In either case (i.e., analog control or digital control), the user must manually increase the intensity of the stimulation to a level that the user believes to be a therapeutic level. Therefore, a major limitation of some TENS devices is that it may be difficult for many users to determine an appropriate therapeutic stimulation intensity. As a result, the user may either require substantial support from medical staff or they may fail to get pain relief due to an inadequate stimulation level.

A newly-developed wearable TENS device (i.e., the Quell® device, Neurometrix, Inc., Woburn, Mass., USA) uses a novel method for calibrating the stimulation intensity in order to maximize the probability that the TENS stimulation intensity will fall within the therapeutic range. With the Quell® device, the user identifies their electrotactile sensation threshold and then the therapeutic intensity is automatically estimated by the TENS device based on the identified electrotactile sensation threshold.

Pain relief from TENS stimulation usually begins within 15 minutes of the stimulation onset and may last up to an hour following the completion of the stimulation period (which is also known as a “therapy session”). Each therapy session typically runs for 30-60 minutes. To maintain maximum pain relief (i.e., hypoalgesia), TENS therapy sessions typically need to be initiated at regular intervals. Newly-developed wearable TENS devices, such as the aforementioned Quell® device, provide the user with an option to automatically restart therapy sessions at pre-determined time intervals.

For TENS users with movement-evoked pain, such as those with fibromyalgia conditions, TENS therapy sessions matching a user's physical activity period are more advantageous than therapy sessions at pre-determined time intervals. By activating TENS therapy automatically during the physical activity period, the TENS device delivers just-in-time relief to the movement-evoked pain. Effective control of movement-evoked pain will allow the TENS user to continue their activities, and thus improve their health conditions.

### SUMMARY OF THE INVENTION

The present invention comprises the provision and use of a novel TENS device which comprises a stimulator designed to be placed on a user's upper calf (or other anatomical location) and a pre-configured electrode array designed to provide electrical stimulation to at least one nerve disposed in the user's upper calf (or other anatomical location). A three-axis accelerometer, either co-located with the TENS device or located in another part of the body, measures the motion and orientation of the user's lower limb in order to continuously and objectively measure the user's activity. A key feature of the present invention is that the novel TENS device automatically controls its operations (e.g., start stimulation, stop stimulation, or change stimulation conditions) according to the aforementioned activity measurements in order to minimize the interference of pain with one or more aspects of quality of life, particularly from the motion-activated pain. Other measurements useful to quantify muscle activities, such as those from electrophysiological sensors (e.g., electromyograph sensors and acoustic myograph sensors), force sensors (e.g., force sensitive resistors), and displacement sensors (e.g., fabric stretch sensors), are also considered as input to control the TENS device operations.

In one form of the invention, there is provided apparatus for providing transcutaneous electrical nerve stimulation (TENS) therapy to a user, said apparatus comprising:

a stimulation unit for electrically stimulating at least one nerve of the user;

a sensing unit for sensing body movement of the user to analyze body movement activity type and activity duration;

an application unit for providing mechanical coupling between said sensing unit and the user's body; and a feedback unit for at least one of (i) providing the user with feedback in response to said analysis of said body movement activity type and activity duration of the user, and (ii) modifying the electrical stimulation provided to the user by said stimulation unit in response to said analysis of said body movement activity type and activity duration of the user.

In another form of the invention, there is provided a method for applying transcutaneous electrical nerve stimulation to a user, said method comprising the steps of:

applying a stimulation unit and a sensing unit to the body of the user;

using said stimulation unit to deliver electrical stimulation to the user so as to stimulate one or more nerves of the user;

analyzing data collected by said sensing unit to determine the user's body movement activity type and activity duration; and

modifying the electrical stimulation delivered by said stimulation unit based on the analysis of body movement activity type and activity duration.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view showing a novel TENS device formed in accordance with the present invention, wherein the novel TENS device is mounted to the upper calf of a user, and also showing the coordinate system of an accelerometer incorporated in the novel TENS device when the user body is in upright and recumbent positions;

FIG. 2 is a schematic view showing the novel TENS device of FIG. 1 in greater detail;

FIG. 3 is a schematic view showing the electrode array of the novel TENS device of FIGS. 1 and 2 in greater detail;

FIG. 4 is a schematic view of the novel TENS device of FIGS. 1-3, including a processor for analyzing activity type, level, and duration, and for analyzing device position;

FIG. 5 is a schematic view showing the stimulation pulse train generated by the stimulator of the novel TENS device of FIGS. 1-4;

FIG. 6 is a schematic view showing the on-skin detection system of the novel TENS device shown in FIGS. 1-5, as well as its equivalent circuits when the novel TENS device is on and off the skin of a user;

FIG. 7 is schematic view showing an example of the accelerometer data waveform from the y-axis of an accelerometer incorporated in the TENS device, with the accelerometer data waveform showing various characteristic events associated with user activity;

FIG. 8 is a schematic view showing exemplary filter operations performed on the exemplary accelerometer data waveform, and the waveform changes due to the filter operations;

FIG. 9 is a schematic view showing processing steps for determining gait variability metrics based on a stride duration time series;

FIG. 10 is a schematic view showing an exemplary coordinate system transformation and its utility to determine the rotational position of the novel TENS device based on forward motion acceleration during an activity period; and

FIG. 11 is a schematic flowchart showing exemplary operation of the novel TENS device, including functionalities for tracking activity type, level, and duration, and device placement position determination.

### DETAILED DESCRIPTION OF THE PREFERRED EMBODIMENTS

### The TENS Device in General

The present invention comprises the provision and use of a novel TENS device which comprises a stimulator designed to be placed on a user's upper calf (or other anatomical location) and a pre-configured electrode array designed to provide electrical stimulation to at least one nerve disposed in the user's upper calf (or other anatomical location). A key feature of the present invention is that the novel TENS device automatically tracks the user's movement and controls stimulation parameters according to activity type, level, and duration derived from the movement tracking results obtained from one or more wearable sensors placed on the user.

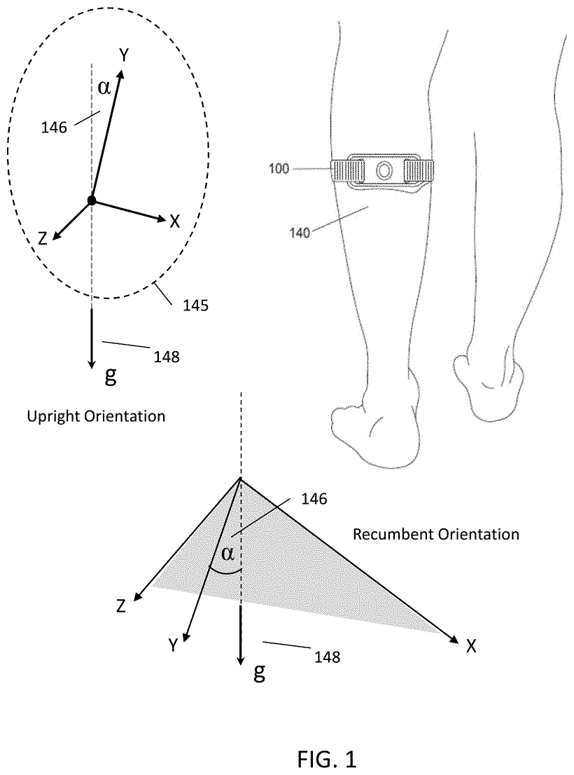

More particularly, and looking now at FIG. 1, there is shown a novel TENS device 100 formed in accordance with the present invention, with novel TENS device 100 being shown worn on a user's upper calf 140. A user may wear TENS device 100 on one leg or on both legs (either one at a time or simultaneously), or a user may wear a TENS device 100 on another area of the body separate from, or in addition to, a TENS device 100 worn on one leg (or both legs) of the user.

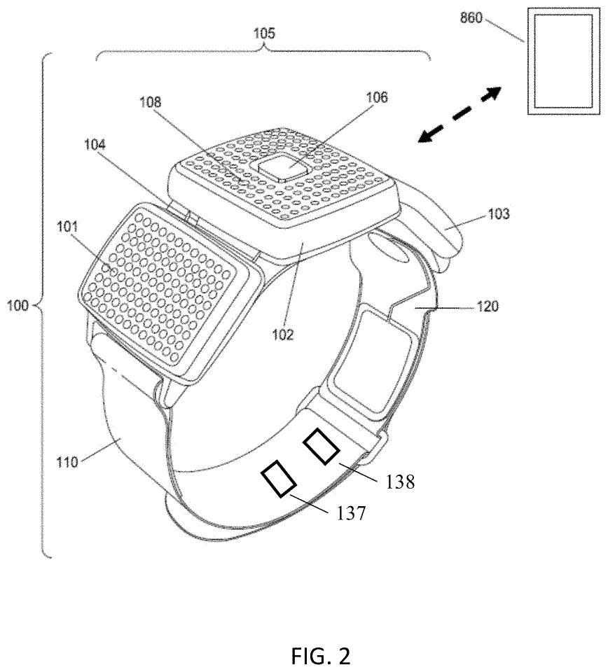

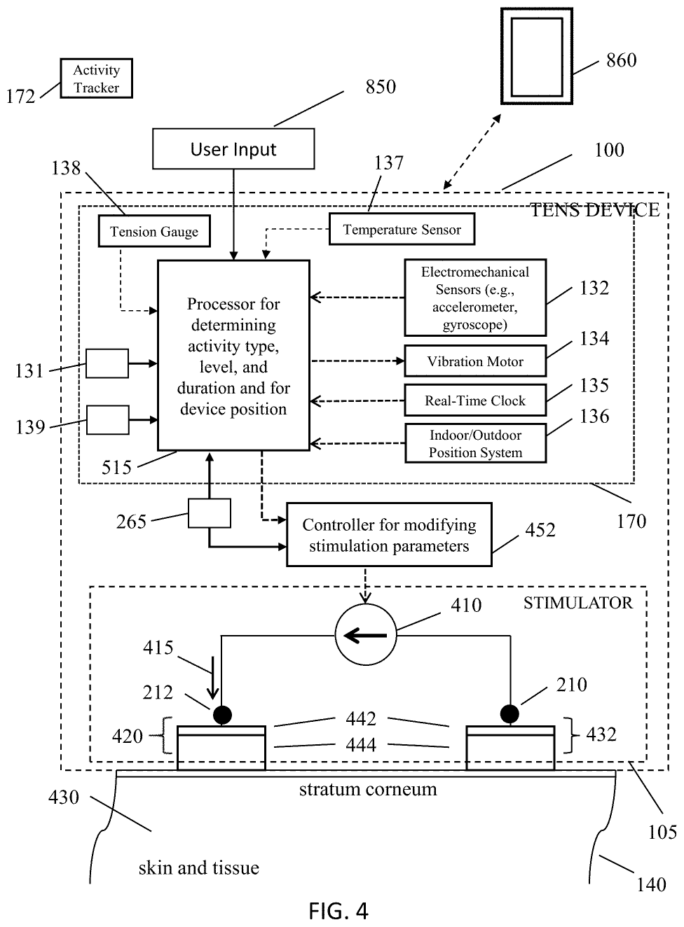

Looking next at FIG. 2, TENS device 100 is shown in greater detail. TENS device 100 preferably comprises three primary components: a stimulator 105, a strap 110, and an electrode array 120 (comprising a cathode electrode and an anode electrode appropriately connected to stimulator 105). As shown in FIG. 2, stimulator 105 may comprise three mechanically and electrically interconnected compartments 101, 102, and 103. Compartments 101, 102, 103 are preferably interconnected by hinge mechanisms 104 (only one of which is visible in FIG. 2), thereby allowing TENS device 100 to conform to the curved anatomy of a user's leg. In a preferred embodiment of the present invention, compartment 102 houses the TENS stimulation circuitry (except for a battery) and user interface elements 106 and 108. Compartment 102 also houses an accelerometer 132 (see FIG. 4), preferably in the form of a MEMS digital accelerometer microchip (e.g., Freescale MMA8451Q), for detecting (i) user gestures such as taps to central compartment 102, (ii) user leg and body orientation, and (iii) user leg and body motion. Compartment 102 also houses a vibration motor 134 (FIG. 4), a real-time clock 135 (FIG. 4), an indoor/outdoor position system 136 (e.g., a global positioning system of the sort typically referred to as a “GPS”), a temperature sensor 137 (FIGS. 2 and 4), and a strap tension gauge 138 (FIGS. 2 and 4).

In one preferred form of the invention, compartments 101 and 103 are smaller auxiliary compartments that house a battery for powering the TENS stimulation circuitry and other circuitry, and other ancillary elements, such as a wireless interface unit (not shown) of the sort well known in the art for allowing TENS device 100 to wirelessly communicate with other elements (e.g., a hand-held electronic device 860, such as a smartphone, see FIG. 2).

In another form of the invention, only one or two compartments may be used for housing all of the TENS stimulation circuitry, battery, and other ancillary elements of the present invention.

In another form of the invention, a greater number of compartments are used, e.g., to better conform to the body and to improve user comfort.

And in still another form of the invention, a flexible circuit board is used to distribute the TENS stimulation circuitry and other circuitry more evenly around the leg of the user and thereby reduce the thickness of the device.

Still looking at FIG. 2, interface element 106 preferably comprises a push button for user control of electrical stimulation by TENS device 100, and interface element 108 preferably comprises an LED for indicating stimulation status and providing other feedback to the user. Although a single LED is shown, interface element 108 may comprise multiple LEDs with different colors. Additional user interface elements (e.g., an LCD display, audio feedback through a beeper or voice output, haptic devices such as a vibrating element, a smartphone running an appropriate “app”, etc.) are also contemplated and are considered to be within the scope of the present invention.

In one preferred form of the invention, TENS device 100 is configured to be worn on the user's upper calf 140 as is shown in FIG. 1, although it should also be appreciated that TENS device 100 may be worn on other anatomical locations, or multiple TENS devices 100 may be worn on various anatomical locations, etc. TENS device 100 (comprising the aforementioned stimulator 105, electrode array 120, and strap 110) is secured to upper calf 140 (or other anatomical location) of the user by placing the apparatus in position against the upper calf (or other anatomical location) and then tightening strap 110. More particularly, in one preferred form of the invention, electrode array 120 is deliberately sized and configured so that it will apply appropriate electrical stimulation to the appropriate anatomy of the user regardless of the specific rotational position of TENS device 100 on the leg (or other anatomical location) of the user.

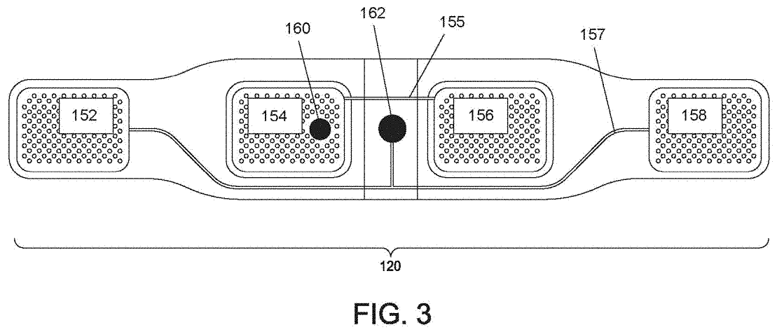

FIG. 3 shows a schematic view of one preferred embodiment of electrode array 120. Electrode array 120 preferably comprises four discrete electrodes 152, 154, 156, 158, each having an equal or similar size (i.e., an equal or similar size surface area). Electrodes 152, 154, 156, 158 are preferably connected in pairs so that electrodes 154 and 156 (representing the cathode of TENS device 100) are electrically connected to one another (e.g., via connector 155), and so that electrodes 152 and 158 (representing the anode of TENS device 100) are electrically connected to one another (e.g., via connector 157). It should be appreciated that electrodes 152, 154, 156, 158 are preferably appropriately sized, and connected in pairs, so as to ensure adequate skin coverage regardless of the rotational position of TENS device 100 (and hence regardless of the rotational position of electrode array 120) on the leg (or other anatomical location) of a user. Furthermore, it should be appreciated that electrodes 152, 154, 156, 158 are not connected in an interleaved fashion, but rather are connected so that the two inside electrodes 154, 156 are connected to one another, and so that the two outside electrodes 152, 158 are connected to one another. This electrode connection pattern ensures that if the two outer electrodes 152, 158 should inadvertently come into contact with one another, an electrical short of the stimulation current flowing directly from cathode to anode will not occur (i.e., the electrode connection pattern ensures that the therapeutic TENS current is always directed through the tissue of the user).

Electrical current (i.e., for therapeutic electrical stimulation to the tissue) is provided to the electrode pairs 154, 156 and 152, 158 by connectors 160, 162 (FIG. 3) which mate with complementary connectors 210, 212 (FIG. 4), respectively, on stimulator 105. Stimulator 105 generates electrical currents that are passed through electrodes 154, 156 and electrodes 152, 158 via connectors 160, 162, respectively.

In one preferred embodiment of the present invention, the skin-contacting conductive material of electrodes 152, 154, 156, 158 is a hydrogel material which is “built into” electrodes 152, 154, 156, 158. The function of the hydrogel material on the electrodes is to serve as an interface between the electrodes 152, 154, 156, 158 and the skin of the user (i.e., within, or adjacent to, or proximal to, the portion of the user's body in which the sensory nerves which are to be stimulated reside). Other types of electrodes such as dry electrodes and non-contact stimulation electrodes have also been contemplated and are considered to be within the scope of the present invention.

FIG. 4 is a schematic representation of the current flow between TENS device 100 and the user. As seen schematically in FIG. 4, stimulation current 415 from a constant current source 410 flows into the user's tissue 430 (e.g., the user's upper calf) via an anode electrode 420 (which anode electrode 420 comprises the aforementioned electrodes 152, 158). Element 410 can also be replaced by a constant voltage source to provide stimulation current 415. Anode electrode 420 comprises a conductive backing (e.g., silver hatch) 442 and hydrogel 444. The current passes through the user's tissue 430 and returns to constant current source 410 through cathode electrode 432 (which cathode electrode 432 comprises the aforementioned electrodes 154, 156). Cathode electrode 432 also comprises a conductive backing 442 and hydrogel 444. Constant current source 410 preferably provides an appropriate biphasic waveform (i.e., biphasic stimulation pulses) of the sort well known in the art of TENS therapy. In this respect it should be appreciated that the designation of “anode” and “cathode” electrodes is purely notational in the context of a biphasic waveform (i.e., when the biphasic stimulation pulse reverses its polarity in its second phase of the biphasic TENS stimulation, current will be flowing into the user's body via “cathode” electrode 432 and out of the user's body via “anode” electrode 420).

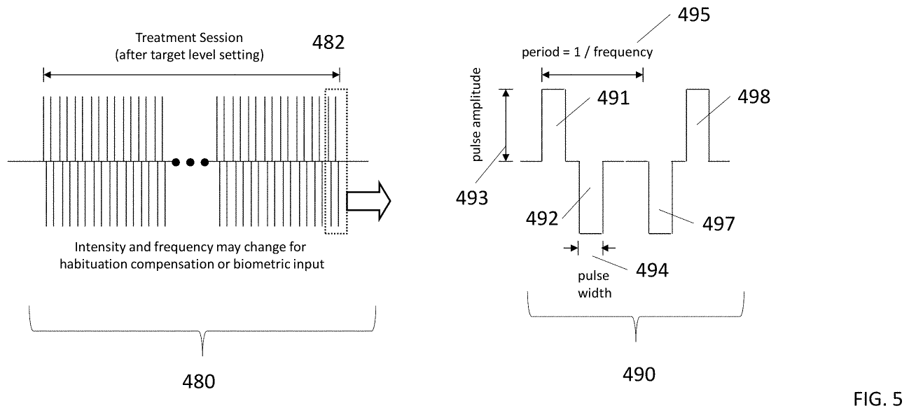

FIG. 5 is a schematic view showing a pulse train 480 provided by stimulator 105 during a TENS therapy session, and the waveform 490 of two individual biphasic pulses, wherein each individual biphasic pulse comprises a first phase 491 or 497 and a second phase 492 or 498. For the first biphasic pulse, the first phase 491 has positive polarity. For the second biphasic pulse, the first phase 497 has negative polarity. In one form of the invention, polarity of the first phase remains the same for all biphasic pulses. In another form of the invention, the first phase of consecutive biphasic pulses alternates its polarity. Yet in another form of the invention, the polarity of the first phase remains positive for one or more biphasic pulses before switching to negative for one or more biphasic pulses. In yet another form of the invention, the first phase of the biphasic pulses randomly switches between positive and negative polarity. In one form of the invention, each pulse waveform is charge-balanced across the two phases 491, 492 (or 497, 498) of the biphasic pulse, which prevents iontophoretic build-up under the electrodes of the electrode array 120 that can lead to skin irritation and potential skin damage. In another form of the invention, the individual biphasic pulses are unbalanced across the two phases of the biphasic pulse, however, charge-balancing is achieved across multiple consecutive biphasic pulses. Pulses of fixed or randomly-varying frequencies are applied throughout the duration of the therapy session 482. The intensity of the stimulation (i.e., the amplitude 493 of the current delivered by stimulator 105) is adjusted in response to user input and for habituation compensation, as will hereinafter be discussed in further detail. The pulse amplitude 493 of the two phases of a biphasic pulse needs not to be the same. Similarly, the pulse width 494 of the two phases needs not to be the same.

Other pulse patterns are also considered. For example, a burst-mode pulse pattern may be employed based on the user's activity monitoring results. As an example, a burst-mode pattern consists of groups of biphasic pulses with the time between each group set at 100 milliseconds. Each group will have 10 biphasic pulses with a pulse period of 2 milliseconds.

In prior U.S. patent application Ser. No. 13/678,221, filed Nov. 15, 2012 by Neurometrix, Inc. and Shai N. Gozani et al. for APPARATUS AND METHOD FOR RELIEVING PAIN USING TRANSCUTANEOUS ELECTRICAL NERVE STIMULATION (Attorney's Docket No. NEURO-5960), issued as U.S. Pat. No. 8,948,876 on Feb. 3, 2015, and which patent is hereby incorporated herein by reference, apparatus and methods are disclosed for allowing a user to personalize the TENS therapy stimulation intensity according to the electrotactile perception threshold of the user at the time of the setup of the TENS device. The aforementioned U.S. Pat. No. 8,948,876 also discloses apparatus and methods to automatically restart additional therapy sessions after an initial manual start by the user.

In prior U.S. patent application Ser. No. 14/230,648, filed Mar. 31, 2014 by NeuroMetrix, Inc. and Shai Gozani et al. for DETECTING CUTANEOUS ELECTRODE PEELING USING ELECTRODE-SKIN IMPEDANCE (Attorney's Docket No. NEURO-64), issued as U.S. Pat. No. 9,474,898 on Oct. 25, 2016, and which patent is hereby incorporated herein by reference, apparatus and methods are disclosed which allow for the safe delivery of TENS therapies at night when the user is asleep. These methods and apparatus allow the TENS device to be worn by a user for an extended period of time, including 24 hours a day.

In order to deliver consistently comfortable and effective pain relief to a user throughout both the day and the night, it may not be appropriate to deliver a fixed TENS stimulation level, since the effect of circadian or other time-varying rhythms can mitigate the effectiveness of TENS stimulation. Parameters impacting TENS stimulation effectiveness include, but are not limited to, stimulation pulse amplitude 493 (FIG. 5) and pulse width 494 (FIG. 5), pulse frequency 495 (FIG. 5), and therapy session duration 482 (FIG. 5). By way of example but not limitation, higher amplitude and longer pulses (i.e., larger pulse charges) increase the stimulation delivered to the user (i.e., the stimulation “dose”), whereas shorter therapy sessions decrease the stimulation delivered to the user (i.e., the stimulation “dose”). Clinical studies suggest that pulse charge (i.e., pulse amplitude and pulse width) and therapy session duration have the greatest impact on the therapeutic stimulation delivered to the user (i.e., the therapeutic stimulation “dose”).

For TENS users with movement-evoked pain, such as those with fibromyalgia, TENS therapy sessions matching a user's physical activity period are more advantageous than therapy sessions at pre-determined time intervals. Therefore, one objective of the present invention is to permit TENS device 100 to automatically adjust its operations based on monitoring the results of the TENS user's movement patterns, or the TENS user's muscle activities, or both. By matching the timing of TENS therapy sessions with that of the events causing the pain, TENS therapy can be more effective in providing pain relief.

User movement has been used to control electrical stimulation. For example, U.S. Patent Publication No. 2010/0004715 (Fahey) and U.S. Patent Publication No. 2010/0217349 (Fahey) disclose a system that delivers electrical stimulation to muscle tissues. The muscle contractions (involuntary body movement) directly caused by the electrical stimulation are then used to adjust stimulation parameters so that certain desired body movement patterns are achieved. The present invention differs from the system disclosed in U.S. Patent Publication No. 2010/0004715 (Fahey) and U.S. Patent Publication No. 2010/0217349 (Fahey) in that movements of a TENS user are voluntary and independent of TENS stimulation and the TENS stimulation does not cause the movement of the user. In the system disclosed in U.S. Patent Publication No. 2010/0004715 (Fahey) and U.S. Patent Publication No. 2010/0217349 (Fahey), modifications of electrical stimulation were based on differences between measured movement patterns and intended movement patterns. The present invention modifies electrical stimulation based on measured movement type, level, and duration without an intended movement pattern as target.

U.S. Patent Publication No. 2013/0158627 (Gozani) provides a general disclosure of using an accelerometer to identify body orientation and the activity of the TENS user and using the identified information to modify the stimulation characteristics in order to optimize stimulation patterns and parameters for the identified state. However, U.S. Patent Publication No. 2013/0158627 (Gozani) does not teach how specific activity type and activity duration can be used to control TENS stimulation to provide relief to the movement-induced pain. In other words, with the system disclosed in U.S. Patent Publication No. 2013/0158627 (Gozani), the simple presence of the activity will trigger changes in TENS stimulation, whereas the present invention will modify TENS operations under specific activity type, level and duration conditions. As an example, the system disclosed in U.S. Patent Publication No. 2013/0158627 (Gozani) will start a TENS therapy session whenever walking activity of the user is detected. The system of the present invention may only start a TENS therapy session when the user has engaged in brisk walking activity for five minutes. If the walk lasts for shorter than five minutes, no TENS therapy will be initiated automatically.

U.S. Patent Publication No. 2016/0144174 (Ferree) discloses a TENS system that monitors specific leg movement patterns known to occur in sleep to determine the sleep state of the user. However, the duration of specific leg movements is not measured or used to control the TENS operations.

U.S. Patent Publication No. 2016/0296935 (Ferree) discloses a TENS system that monitors specific body movement patterns and uses the presence of such movement patterns to permit or to suppress other measurements (e.g., user gesture and electrode-skin contact degradation) to control the TENS device operations.

U.S. Patent Publication No. 2014/0309709 (Gozani) discloses a TENS system that monitors activity level and body orientation of the TENS user during sleep. If the activity level remains low and body orientation remains recumbent for a period of time, the TENS stimulation parameters may be modified. With the system of U.S. Patent Publication No. 2014/0309709 (Gozani), automated control of TENS operations is conditioned up both recumbent body orientation and lack of body activities of any kind for a period time. In contrast, the present invention controls TENS operations based on the presence of body activities of specific patterns for a period of time.

U.S. Patent Publication No. 2017/0312515 (Ferree) discloses a TENS system similar to that of U.S. Patent Publication No. 2014/0309709 (Gozani). In addition to body orientation and body movement (activity level), a specific activity pattern (periodic leg movement, or PLM) is monitored. Only the occurrence count of the PLM is used, in conjunction with body orientation and body movement, to control the TENS stimulation. The PLM duration is not measured nor used in TENS stimulation control.

U.S. Patent Publication No. 2018/0132757 (Kong) discloses a TENS system that monitors biomarkers such as activity level, gait, and balance of users wearing a TENS device to objectively assess the benefits of TENS therapies. The system also uses those monitored biomarkers to automatically adjust TENS operations. However, only the activity level is used to control TENS operations, and activity duration is not used in TENS stimulation control.

U.S. Patent Publication No. 2013/0116514 (Kroner) disclosed a seizure detection system that detects certain body movement patterns (i.e., those of seizure type) and issues an alert. However, the alert action is immediate upon the detection of a pre-defined activity type, without any consideration of the activity duration.

Assessments of the therapeutic benefits of TENS therapy are often subjective, infrequent, and incomplete, such as those measured by responses to clinical questionnaires or pain diaries. Furthermore, the perception of pain (i.e., the subject's self-evaluation of pain levels) is only one of many important dimensions of effective pain relief. More active lifestyle, steadier gait, and better balance are important examples of improved quality of life and health. These improvements can be attributed to a reduction of pain as a result of TENS therapy. In prior U.S. Patent Publication No. 2018/0132757 (Kong), apparatus and methods are disclosed which provide one or more biomarkers that are objectively and automatically measured and are based on assessing the activity, gait, and balance of the user wearing a TENS device. Apparatus and methods are also disclosed to permit a TENS device to automatically adjust its operations based on the results obtained from monitoring the activity level, gait, and balance of the user.

While there is strong evidence to suggest that physical activity is an effective treatment for fibromyalgia, many individuals with fibromyalgia report that movement-evoked pain limits their activities. If movement-evoked pain can be reduced, individuals with fibromyalgia will be able to engage in longer and more robust physical activities as prescribed by their caregivers, resulting in an improvement of overall health to these individuals. TENS therapy has been shown to be effective in reducing movement-evoked pain of fibromyalgia patients in a clinical study (Dailey et al 2020). In the study, participants were instructed to use TENS therapy during their physical activity. However, TENS therapies were manually initiated by the study participants.

To maximize pain relieving effects of TENS therapy for individuals with fibromyalgia, it is desirable to initiate TENS therapy automatically only after physical activities are detected. Because fibromyalgia patients only experience movement-evoked pain after a certain period of physical activity, TENS therapy should be activated only after continuous physical activity has been detected for a minimum time period. More rigorous activity could result in a higher level of pain. Therefore, the onset and intensity level of TENS therapy should also be controlled automatically based on the combined effect of activity level and activity duration. With the present invention, apparatus and methods are disclosed to control TENS therapy operations based on the TENS user's physical activity type, activity level and/or activity duration to reduce movement-evoked pain.

Examples of types of exercises that are beneficial to fibromyalgia patients include fast walking and cycling. These activities and other movement-related activities can be monitored and measured by electromechanical sensors such as accelerometers. Strengthening and stretch exercises are also recommended activities for fibromyalgia patients. Although these exercises may not be correlated with significant body movements, they do require muscle activities such as muscle contractions and relaxation. These muscle activities can be monitored and measured by non-invasive and wearable sensors such as a stretchable conductive rubber sensor (P. Bifulco et al., “A stretchable, conductive rubber sensor to detect muscle contraction for prosthetic hand control,” 2017 E-Health and Bioengineering Conference (EHB), Sinaia, Romania, 2017, pp. 173-176, doi: 10.1109/EHB.2017.7995389), force sensitive sensors or fabric stretch sensors (O. Amft et al., “Sensing muscle activities with body-worn sensors. Int Work Wearable Implant Body Sens Networks,” 2006, 10.1109/BSN.2006.48), an electromyography (EMG) sensor, or an acoustic myography (AMG) sensor.

In one preferred embodiment, an activity tracker (the element that determines activity type, level, and duration) is embedded in the same housing as the stimulator as a part of the TENS system. In another embodiment, an activity tracker is co-located with the stimulator on the user but in a different housing. In yet another embodiment, an activity tracker is located at a different anatomic location of the user. Unless specific stimulation functions are cited, the terms activity tracker and TENS device are sometimes used interchangeably in this application.

Overview of Invention

Generally, physical activities are either associated with upright body orientation (such as walking) or with recumbent body orientation (such as strength and stretch exercise). Various elements of the automated TENS control apparatus based on user activity level and duration are described in the following paragraphs. An on-skin detector establishes the physical coupling of an activity tracker (as a part of the TENS system or as a separate element) and the user body to correlate body orientation and movement with patterns of measurements from one or more sensors embedded in the activity tracker. One such example of sensors is a three-axis accelerometer. Next the alignment between accelerometer axes and body axes is determined by leveraging the gravitational force and two other elements of the TENS system: Device Orientation Determination and Vertical Alignment Compensation. Walking is the most commonly engaged physical activity when the body orientation is upright. A Walk Activity Level and Duration Determination section provides a detail description of apparatus and methods to automatically quantify walking, the most common physical activity. A Cycling Activity Determination section provides a detail description of apparatus and methods to automatically quantify cycling, another common physical activity. An Other Activity Determination section provides a description of apparatus and methods to automatically quantify other physical activities such as strength, stretching, and isometric exercises. A Controller For Modifying Stimulation Parameters section details apparatus and methods for controlling TENS operations based on activity type, level, and duration as measured by the activity tracking element. Finally, an exemplary operation of the invention is given in Exemplary Operation section.

On-Skin Detector

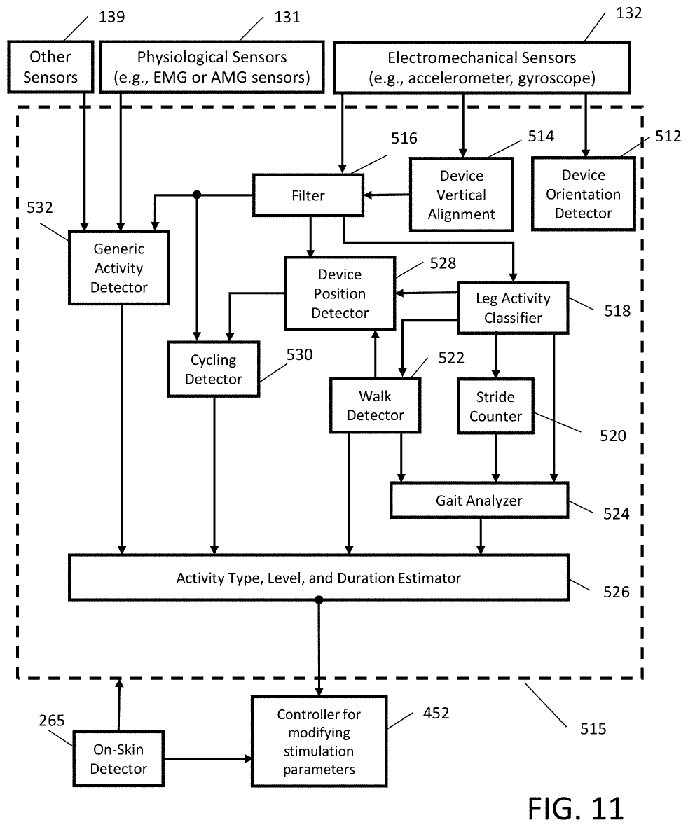

In one preferred form of the invention, TENS device 100 may comprise an on-skin detector 265 (FIGS. 4 and 11) to confirm that TENS device 100 is firmly seated on the skin of the user.

More particularly, the orientation and motion measures from accelerometer and/or gyroscope 132 (FIG. 4) of TENS device 100 only become coupled with the orientation and motion of a user when the TENS device is secured to the user. In a preferred embodiment, an on-skin detector 265 (FIG. 4) may be used to determine whether and when TENS device 100 is securely placed on the user's upper calf.

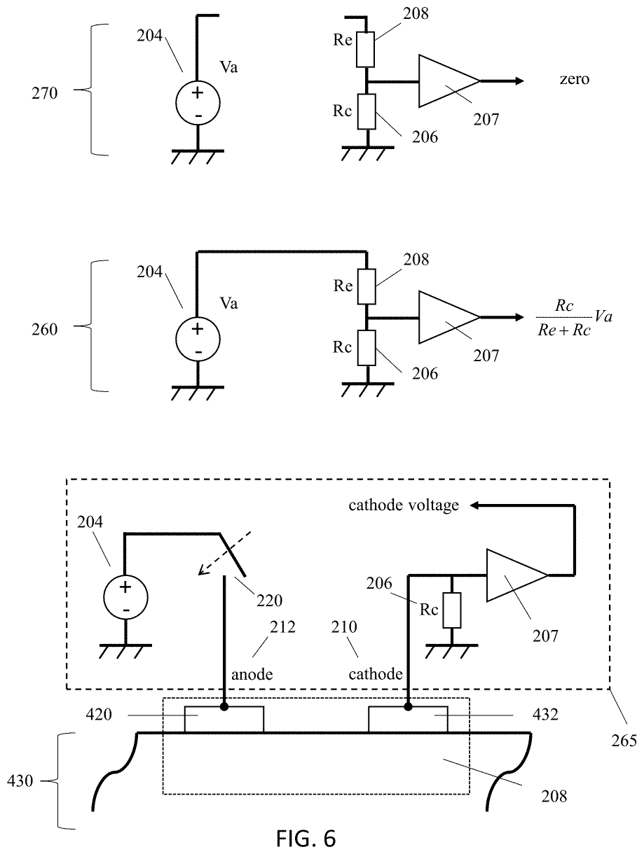

In the preferred embodiment, and looking now at FIG. 6, an on-skin detector 265 may be incorporated in TENS device 100. More particularly, in one preferred form of the invention, a voltage of 20 volts from voltage source 204 is applied to anode terminal 212 of TENS stimulator 105 by closing the switch 220. If the TENS device is worn by the user, then user tissue 430, interposed between anode electrode 420 and cathode electrode 432, will form a closed circuit to apply the voltage to the voltage divider circuit formed by resistors 208 and 206. More particularly, when TENS device 100 is on the skin of the user, the equivalent circuit 260 shown in FIG. 6 represents the real-world system and equivalent circuit 260 allows the anode voltage Vₐ 204 to be sensed through the voltage divider resistors 206 and 208. The cathode voltage measured from the amplifier 207 will be non-zero and close to the anode voltage 204 when TENS device 100 is secured to the skin of the user. On the other hand, when TENS device 100 is not secured to the skin of the user, the equivalent circuit 270 represents the real-world system and the cathode voltage from amplifier 207 will be zero.

On-skin detector 265 is preferably employed in two ways.

First, if on-skin detector 265 indicates that electrode array 120 of TENS device 100 has become partially or fully detached from the skin of the user, TENS device 100 can stop applying TENS therapy to the user.

Second, if on-skin detector 265 indicates that electrode array 120 of TENS device 100 has become partially or fully detached from the skin of the user, processor 515 (FIG. 4) of TENS device 100 will recognize that the data from accelerometer and/or gyroscope 132 may not reliably reflect user leg orientation and leg motion. In this respect it should be appreciated that when the on-skin detector 265 indicates that TENS device 100 is secured to the skin of the user, such that accelerometer and/or gyroscope 132 is closely coupled to the lower limb of the user, the data from accelerometer and/or gyroscope 132 may be considered to be representative of user leg orientation and user leg motion. However, when the on-skin detector 265 indicates that TENS device 100 is not on the skin of the user, accelerometer and/or gyroscope 132 is not closely coupled to the lower limb of the user, the data from accelerometer and/or gyroscope 132 cannot be considered to be representative of user leg orientation and user leg motion.

An on-skin condition is necessary for the TENS device to stimulate the user inasmuch as a closed electrical circuit is needed for the stimulation current to flow. However, the on-skin condition is not necessary for the TENS device to monitor the user activity. The TENS device can still perform these monitoring functions and determine placement position of the TENS device as long as the device is positioned on the body.

In one preferred form of the invention, a strap tension gauge 138 (FIGS. 2 and 4) on the TENS device measures the tension of the strap 110. When the strap tension meets a pre-determined threshold, the TENS device 100 is considered “on-body” and the monitoring functions can continue even if the on-skin condition may not be met. In another embodiment, the tension gauge value while the on-skin condition is true is used as the on-body tension threshold. When the on-skin condition becomes false, as long as the tension gauge value is above the on-body tension threshold, the on-body status remains true. All activity monitoring functions can still be performed as long as the on-body status is true. Furthermore, position of the TENS device placement on the body can also be performed as long as the on-body status is true.

In one preferred form of the invention, a temperature sensor 137 (FIGS. 2 and 4) incorporated in the TENS device 100 measures the skin temperature and the skin temperature measurement is used to determine on-body status of the TENS device 100. In a preferred embodiment, the skin temperature measurements during the on-skin condition are averaged and stored as a reference. When the on-skin condition transitions from true to false, the skin temperature is continuously monitored. If the measured skin temperature remains similar to the reference skin temperature, the on-body status is set to true to indicate that the TENS device 100 is still on the user's body. Consequently, all activity monitoring functions can still be monitored. Furthermore, a determination of the position of the TENS device placement on the body can also be performed as long as the on-body status is true.

Accelerometer Data Sampling

In one preferred form of the invention, TENS device 100 samples accelerometer 132 at a rate of 400 Hz, although a different sampling rate can be utilized.

Device Orientation Determination

In one preferred form of the invention, TENS device 100 (comprising accelerometer 132) is strapped on a user's upper calf 140, e.g., in the manner shown in FIG. 1. The three axes of the accelerometer 132 are shown in FIG. 1 as well. The y-axis of accelerometer 132 is approximately aligned with the anatomical axis of the leg, thus the gravitational force g 148 (“gravity” for short) is approximately parallel to the y-axis of accelerometer 132 when the user is standing. When TENS device 100 is placed on the leg with an “upright” orientation, accelerometer 132 will sense an acceleration value of −g, but when TENS device 100 is placed on the leg with an “upside down” orientation, accelerometer 132 will sense an acceleration value of +g.

In one preferred embodiment, the orientation of TENS device 100 is assessed through device orientation detector 512 (FIG. 11) once on-skin detector 265 determines that TENS device 100 is “on-skin”. The y-axis values of accelerometer 132 are accumulated for a period of ten seconds, and then the mean and standard deviation for the y-axis values are calculated. If the standard deviation is below a pre-determined threshold, it suggests that the user has had no activities during that time period (i.e., the ten second time period under review). The mean value is checked against a set of pre-determined threshold values. If the mean value is smaller than −0.5*g, then the device orientation is deemed to be upright. If the mean value is larger than +0.5*g, then the device orientation is deemed to be upside down. If the mean value (i.e., acceleration along the y-axis) is between −0.5 g and +0.5 g, the leg is likely to be in a recumbent position and the device orientation cannot be reliably determined. In this case, a new set of y-axis values will be collected and the above process repeated until the device placement orientation can be reliably determined. Once the device placement orientation is determined, the orientation status of the device stays the same (i.e., upright or upside down) until the on-skin condition becomes “false” (i.e., until the TENS device is determined to no longer be “on-skin”) and the device placement orientation returns to an undefined state.

In one preferred form of the invention, the on-skin status will also set the on-body status to true.

Temperature sensor 137 and tension gauge 138 can be used to assess the on-body status as disclosed earlier. When the on-skin status becomes “false” due to the loss of electrical contact between the TENS device 100 and the user's skin, the on-body status is assessed based on measurements from temperature sensor 137 or tension gauge 138 or both. The measurement values are compared with a fixed reference threshold or a threshold established during the on-skin period. The device placement orientation status is maintained as long as the on-body status is true.

In one preferred form of the invention, accelerometer measurements acquired from a TENS device placed upside down are mapped to values as if they were collected from a TENS device placed upright in order to simplify data analysis for subsequent activity level and duration assessment. In another embodiment, the data analysis methods are developed separately for data acquired under the two different device orientations (i.e., device upright and device upside down).

In one preferred form of the invention, the activity level and intensity assessments (see below) are not performed until the device orientation is determined. In another form of the invention, the assessments are performed under the assumption that the device orientation is upright when the device orientation state is undefined. Results obtained under such an assumption are adjusted if the actual device orientation is later determined to be upside down. In yet another form of the invention, the assessments are performed under the assumption that the device orientation is the same as the device orientation determined in a previous on-skin session. In yet another form of the invention, the assessments are performed under the assumption that the device orientation is the same as the majority of device orientations observed in the past. Regardless of the basis of the assumptions, once the actual device orientation is determined, the activity level and duration assessment results are adjusted as needed.

For the sake of clarity, subsequent descriptions will assume that the device placement orientation is upright or that the accelerometer data are mapped to values corresponding to an upright device placement.

Vertical Alignment Compensation

Under the ideal condition (i.e., upright device placement, no external movements such as those experienced on a traveling train, etc.), the y-axis signal from accelerometer 132 stays at the −1*g level (i.e., the static acceleration value caused by earth gravity) when a subject is standing still. The y-axis acceleration value from accelerometer 132 goes above and below this value depending upon leg activities. However, the relative position of the y-axis direction of accelerometer 132 and the direction of earth gravity may not be perfectly aligned (e.g., due to leg anatomy and device placement variations) so the zero activity acceleration value may be different from −1*g.

To determine the exact alignment relationship between the y-axis of accelerometer 132 and earth gravity direction ((α 146 in FIG. 1), each time TENS device 100 is placed on the leg of a user (and the “on-skin” condition transitions from false to true), an automated calibration algorithm is preferably used to determine and compensate for any misalignment between the directions of the y-axis of accelerometer 132 and earth gravity. The axes 145 of the accelerometer 132 are shown in FIG. 1. This automated calibration algorithm is shown as device vertical alignment unit 514 in FIG. 11.

In the preferred embodiment, an initial segment of accelerometer data corresponding to the user standing upright (i.e., the y-axis acceleration mean yₘₑₐₙ value being greater than a pre-determined threshold) and the user being still (i.e., the y-axis acceleration standard deviation y_{ztdev} value smaller than a pre-determined threshold) is analyzed to determine an average of the static gravitational acceleration value. This value is compared with the expected static gravitational acceleration value and the angle (a 146 in FIG. 1) between the two axis directions (i.e., the y-axis acceleration of accelerometer 132 and earth gravity g) can be calculated. The angle α 146 (which essentially identifies misalignment between the y-axis of accelerometer 132 and earth gravity) is then used to compensate for any effects of misalignment of these two axes.

In one preferred form of the invention, the acceleration values from the y-axis of accelerometer 132 are accumulated over a period of ten seconds and the mean is calculated: this value is defined as yₘₑₐₙ. The angle α 146 (FIG. 1) between the y-axis of accelerometer 132 and the gravity g 148 (FIG. 1) can be estimated with the formula α=cos ⁻¹ yₘₑₐₙ/g).

In another embodiment, multiple estimates of the angle α 146 are averaged and used in subsequent data analysis.

With the knowledge of the estimated angle α 146, one can determine leg orientations of the TENS user. If the angle between earth gravity g and x-z plane of the accelerometer is close to the estimated angle, the leg orientation is in a recumbent position. A person will have a recumbent leg orientation when the person is lying comfortably in bed or on an exercise mat on the floor.

It is often desirable to remove the static gravitational acceleration value from the raw accelerometer measurements before the activity-related analyses are performed. Once the leg orientation is NEURO-107 determined to be upright, static gravitational force −g can be removed from the y-axis accelerometer measurement. Alternatively, instead of removing −g from the y-axis accelerometer measurement, the exact projection of the static gravitation acceleration −g*cos(a) is removed to improve the accuracy of the activity-related assessments. The purpose of this approach is to obtain a better reference to the zero-activity level for the accelerometer data.

Similarly, if the leg orientation is determined to be recumbent, static gravitational force −g or −g*cos(a) can be removed from the accelerometer projection on the x-z plan to improve the accuracy of the activity related assessments.

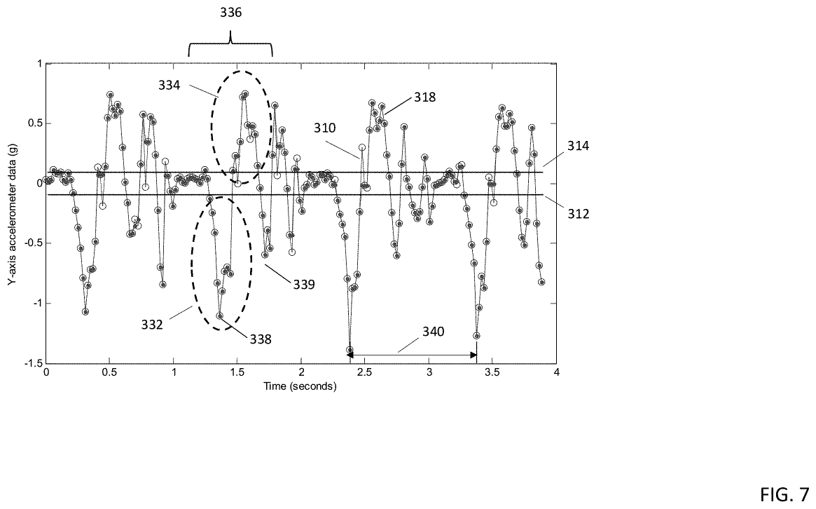

Background noise may cause the y-axis acceleration values of accelerometer 132 to fluctuate around the zero-activity level after the static gravity value is removed. To compensate for background noise, two times the standard deviation y_{ztdev} (see above) is added to, and subtracted from, this zero-activity level in order to create a “zero-activity band”. In the preferred embodiment, although the device orientation will only be determined one time for each device “on-skin” session, this zero-activity band is updated whenever a new estimation of {yₘₑₐₙ, y_{ztdev}} becomes available. The upper bound 314 (FIG. 7) of the zero-activity band is referred to as the “positive zero-crossing threshold” and the lower bound 312 (FIG. 7) of the zero-activity band is referred to as the “negative zero-crossing threshold”.

As an example, when a TENS user resting comfortably on an exercise mat engages in a leg stretching exercise, data measured from the accelerometer will have the following measurement patterns: 1) gravitational acceleration −g or −g*cos(a) is detected within the x-z plane of the accelerometer; 2) motion along the y-axis of the accelerometer and such motion follows a pattern of a period function approximately as the user repeats the leg stretching activity.

Walk Activity Level and Duration Determination

Filtering Operation

Filtering operations are designed to preserve waveform features critical to activity analysis while suppressing noise and other inconsequential features. The filter unit 516 (FIG. 11) takes input from accelerometer 132 and setup parameters from device vertical alignment unit 514 to produce output suitable for further processing by leg activity classifier unit 518 (FIG. 11).

Walking is the most common form of physical activity. We describe in detail below how the walking activity is recognized with an accelerometer that is mechanically coupled with a leg. Repeated leg swing motion is a signature of walking. Looking now at FIG. 7, the open circles connected with dotted lines 310 represent the accelerometer y-axis values after the gravity bias yₘₑₐₙ has been removed. The two horizontal lines are the negative zero-crossing threshold 312 and the positive zero-crossing threshold 314. The solid discs connected with solid lines 318 (overlapping lines 310 in many samples) are the filtered accelerometer y-axis values.

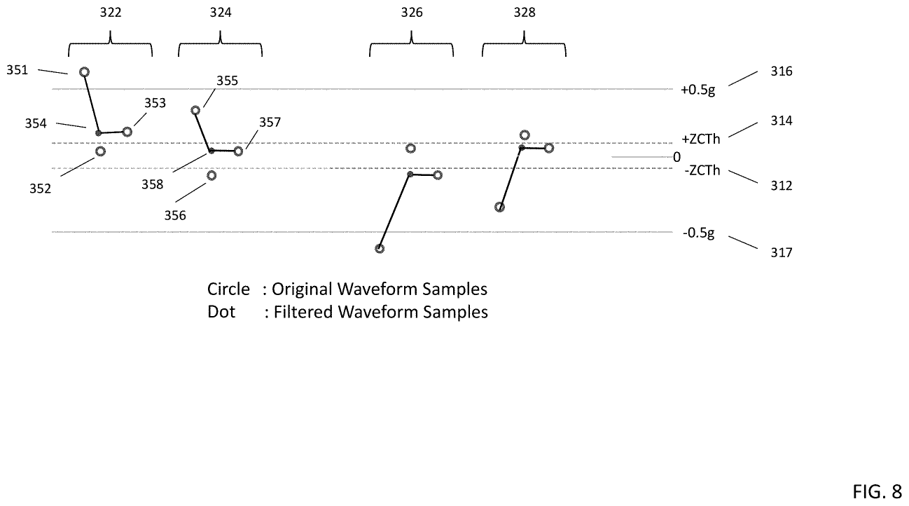

In one preferred embodiment, a selective “median” filter is used to filter the original accelerometer data. The effect of the median filter can be seen in FIG. 7 on waveform samples near or within the zero-activity band (i.e., the region between thresholds 312 and 314) while waveform samples with a larger amplitude are not affected. The median filter is applied selectively to individual waveform samples based on its immediate neighbor sample magnitude. FIG. 8 illustrates the four cases when waveform samples are subject to the median filter operations. The median filter operates on one waveform sample at a time. In case 322, original waveform sample 352 is subject to the median filter operation. The filter examines the two immediate neighboring samples 351 and 353. One of samples 351 has a large amplitude outside the boundary line 316 (e.g., +0.5*g). The filter modifies (i.e., filters) the sample 352 by changing its amplitude to the median of the original amplitude of the three samples 351, 352, and 353. In this case, the median value is that of sample 353. Therefore, the output of the selective median filter for sample 352 will be 354, taking the amplitude value of 353. Median filter operations for case 326 work similarly as that for case 322. In case 324, current waveform sample 356 and its immediate neighbors 355 and 357 are all within a region bounded by boundary line 316 (e.g., +0.5*g) and 317 (e.g., −0.5*g). However, the transition from sample 355 to sample 356 causes waveform to cross the zero activity region (from above to below the region). Additionally, the amplitude difference between the current sample 356 and either neighbor sample exceeds a threshold 0.75*g. Under these conditions, the filter modifies the amplitude of the current sample 356 to the median of the original amplitudes of the three samples 355, 356, 357. In this case, the median value is that of sample 357. Therefore, the output of the selective median filter for sample 356 will be 358. Median filter operations for case 328 work similarly as that for case 324. In other cases, the current sample retains its original amplitude value. It is noted that a threshold crossing event could still occur even after application of the median filter depending upon the exact value of the neighboring sample points. It is also noted that the values of +0.5*g (which is used to set boundary line 316), −0.5*g (which is used to set boundary line 317), and 0.75*g (which is used to help determine applicability of median filter operations on the current sample) are those chosen for one preferred form of the invention, other values may be used and are considered to be within the scope of the present invention.

Swing Event Identification

Leg activity identifier unit 518 (FIG. 11) identifies leg swing events based on specific characteristics of accelerometer waveforms. The following characteristics are evident for the filtered y-axis accelerometer data waveform 318 (FIG. 7) associated with a leg swing event 336 (i.e., a stride) (FIG. 7) when the user is making a stride: a segment (negative phase, 332 in FIG. 7) of the waveform is below the negative zero-crossing threshold 312, followed immediately by a larger segment (positive phase, 334 in FIG. 7) of the waveform being above the positive zero-crossing threshold 314. Areas of the positive and negative phases are calculated. For the purpose of area calculation, the magnitude of each sample is limited to 1*g to minimize the effect of large acceleration spikes. The area of the smallest rectangle that covers the magnitude-limited positive phase (i.e., “the positive rectangular area”) is also calculated. A stride (e.g., leg swing event 336 in FIG. 7) is recognized if all of the following conditions are met:

1. the positive phase duration is no greater than a first threshold Th1;

2. the positive phase duration is no shorter than a second threshold Th2;

3. the swing event is not too close to a previously-detected swing event (i.e., the difference in the timings of the two events is greater than a pre-determined threshold);

4. the area of the positive phase (334 in FIG. 7) is no smaller than a third threshold Th3;

5. the “positive rectangular area” is no smaller than a fourth threshold Th4, or the combined area of the positive and negative phases (332 and 334 in FIG. 7) is no smaller than 1.5 times the threshold Th4; and

6. the maximum amplitude of the positive phase (334 in FIG. 7) is no smaller than a fifth threshold Th5, or the peak-to-peak amplitude (i.e., the positive phase peak waveform value minus the negative phase peak waveform value) is no smaller than a sixth threshold Th6.

Each leg swing event 336 (FIG. 7) which is identified adds one stride to a stride count (which is recorded in a counter or register) through a stride counter 520 (FIG. 11). The step count is defined as twice the stride count for any measurement period. The timing for each stride is anchored to a “toe-off” event, which is the time instance 338 (FIG. 7) associated the valley of the waveform 318. The “toe-off” event corresponds to the time instance when one foot is moving off the ground immediately prior to the swinging of the leg forward. The time difference between two consecutive toe-off events (340 in FIG. 7) is called the stride duration if the time difference is below a threshold (e.g., 3 seconds). Cadence is calculated by dividing the step count by the time interval corresponding to the steps taken.

In another embodiment, gyroscope data (from gyroscope 132, FIG. 4) are used to detect and quantify leg swing activities. Gyroscope 132, incorporated in TENS device 100 (which is attached to the leg of the user), can measure the angular acceleration and velocity of the leg during leg swing periods.

WalkNow Status Indicator

In one preferred form of the invention, TENS device 100 also comprises a walk detector 522 (FIG. 11) to set the “WalkNow status indicator”. The WalkNow status indicator is set to FALSE by default. When five or more strides are detected, the average stride duration is calculated if no two consecutive strides are separated by more than a pre-determined threshold time interval (e.g., 5 seconds). If the average stride duration is no greater than the pre-determined threshold time interval, then the WalkNow status indicator is set to TRUE. If at any time two consecutive strides are separate by more than the threshold time interval, then the WalkNow status indicator is reset to FALSE. When the WalkNow status indicator is set to TRUE, the Activity type, level, and duration unit 526 registers activity type as walk and sets activity duration as the time duration the WalkNow status is true.

Gait Analysis

The primary objective of gait analysis is to assess and characterize gait variability. Gait variability is an effective predictor of fall risk (Hausdorff et al, Gait variability and fall risk in community-living older adults: a 1-year prospective study. Arch Phys Med Rehabil., 2001; 82(8):1050-6). In one preferred form of the invention, stride duration variability is measured. Stride durations are obtained when the TENS user is in his or her natural walking environment. This is in contrast to most gait variability measurements that are done in a laboratory setting. A coefficient of variation (CoV) value is calculated for each qualified walk segment. A walk segment is a sequence of consecutive strides when the WalkNow status remains true. A qualified walk segment is a walk segment whose stride characteristics meet certain criteria, such as the number of strides exceed a minimum threshold. Because the walking environment may influence gait variability, the daily distribution of CoV (percentile values) is updated and reported to the user whenever a qualified walk segment becomes available. The major functional blocks of gait analyzer unit 524 (FIG. 11) include:

1. toe-off event detection;

2. gait segment determination; and

3. gait variability estimation.

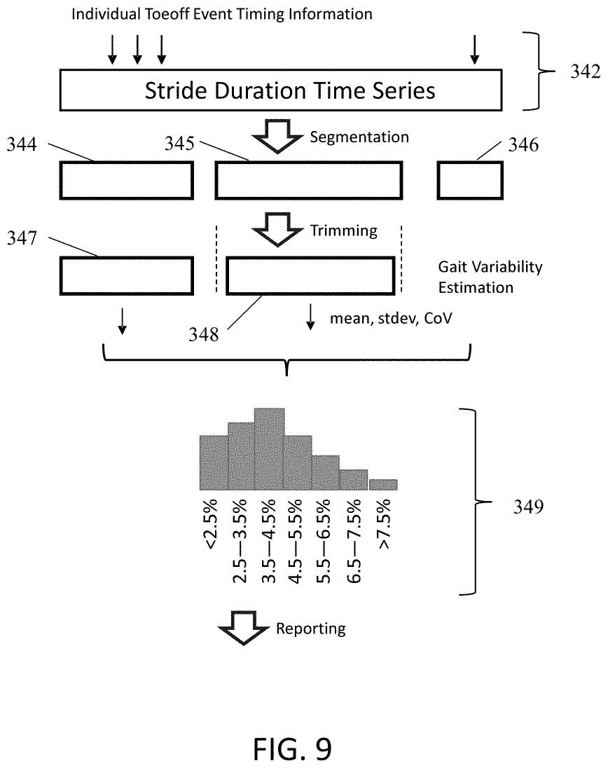

A flowchart summarizing gait analysis is shown in FIG. 9.

Toe-Off Event Timing Detection

Walking involves periodic movements of legs. Any readily identifiable event of leg movement can be used to mark the period of the periodic movements (stride duration). Two events, the “heel strike” and “toe-off” events, are commonly used for stride duration estimation and gait variability analysis. The “heel strike” event is the time instance when the heel of a foot makes the initial contact with the ground during walk. The “toe-off” event corresponds to the time instance when a foot is moving off the ground immediately prior to the swinging of the leg forward. In one preferred embodiment, toe-off events are used in gait analysis. Exact toe-off event timing is traditionally obtained through examining force-mat or force sensor measurements. However, measurements from accelerometer 132 incorporated in the TENS device (which is attached to upper calf of the user) provide distinct features that are highly correlated with actual toe-off events. In one preferred form of the invention, the timing of negative peaks 338 (FIG. 7) prior to the positive phase 334 (FIG. 7) are used to approximate the timing of the toe-off events. Although the timing of negative peaks 338 may not coincide precisely with the actual toe-off time, the relationship between the two is strong and provide a high correlation. Stride durations derived from a force-sensor (for actual toe-off events) and those derived from accelerometer 132 using negative peaks 338 also exhibit very high correlation under various gait conditions (e.g., walk at normal pace, walk at faster pace, walk at slower pace, etc.).

Once a stride (336, a positive phase 334 following a negative phase 332) is detected, recorded negative peaks 338 are examined within a time window prior to the stride detection event. In one preferred embodiment, the negative peak 338 with the largest amplitude is identified and its timing is used as the toe-off event time. If no negative peak 338 exists within the search window, then the timing of the negative peak 338 that is closest to stride detection event is used.

In yet another embodiment, similar features of the accelerometer signal from an axis other than the y-axis are used to determine toe-off events. The difference between two consecutive toe-off events is recorded as a stride duration.

Stride Duration Series Segmentation

Stride duration time series 342 (FIG. 9) is accumulated for the duration of each walk segment. If the number of stride duration measurements exceeds a maximum count, the stride duration series is divided into a plurality of segments (each up to the maximum count). In one preferred embodiment, the mean and standard deviation for each segment of the stride duration series are calculated and an outlier threshold is set based on calculated mean and standard deviation values. Stride durations are flagged as outliers if the absolute values of the differences from the mean exceed the outlier threshold. These outliers, if any, divide the original series into smaller segments of consecutive stride durations for gait variability assessment. FIG. 9 shows three such segments 344, 345, and 346 derived from a stride duration time series 342.

Stride Duration Segment Trimming

Still looking at FIG. 9, for each segment having a segment length (segment length is the number of stride durations in the segment) exceeding a minimum segment length (e.g., 30 strides), the segment becomes an eligible gait variability assessment segment 345. Statistics of the duration time series are calculated for each eligible gait segment. Before calculation, the first and last five stride duration samples of the segment are temporally trimmed to form a middle segment. The maximum absolute difference of the samples from the middle segment mean is calculated. The middle segment is then expanded, sample by sample, to include contiguous adjacent samples from the first five until the sample difference from the mean exceeds the maximum absolute difference. The expansion to include durations from the last five samples proceeds similarly. As a result of this operation, each segment 347 (FIG. 9) and 348 (FIG. 9) contains a series of stride durations suitable for gait variability estimation.

Gait Variability Estimation

For each eligible segment 347 and 348, the mean and standard deviation values of the stride duration samples are calculated. The coefficient of variation (CoV) is also calculated. In one preferred embodiment, the daily minimum CoV is maintained for each user as the gait variability metric. In another embodiment, the gait variability metric is a histogram 349 (FIG. 9) of the CoV (in percentage values) with the following bins: <2.5%, 2.5%-3.5%, 3.5%-4.5%, 4.5%-5.5%, 5.5%-6.5%, 6.5%-7.5%, and >7.5%. The gait variability metrics are reported through a gait variability reporter unit 526 (FIG. 11) to the user whenever an eligible gait analysis segment becomes available. In another embodiment, gait variability metrics is reported under different step cadence conditions. For example, gait variability of slow leisure walking is reported separately from the gait variability of brisk walking.

In prior U.S. Patent Publication 2018/0132757 (Kong), the daily minimum CoV was used to determine the inherent gait variability of the TENS user. A minimum CoV represents the best performance (limit) of the user's ability to maintain a steady gait under any walking conditions. In one preferred embodiment of the present invention, segment-by-segment CoV values (e.g., those associated with 347 and 348) are used to determine the walking activity difficulty levels by feeding the results of Gait Analyzer 524 to Activity Type, Level, and Duration Estimator 526. The estimator 526 can then determine the activity level based on CoV values. For example, when a user is walking on a paved sidewalk, the CoV value for that walk segment will be lower than the CoV value for a walk segment on a hiking trail. The effort involved in making same number of steps on the hiking trail is greater than the effort needed on the paved sidewalk. Therefore, gait variability as measured by the CoV can be used to modify the duration of the physical activities such as walking. The advantage of considering both duration and effort of an activity type is that the exertion on the user's muscles can be more accurately estimated. Movement-evoked pain can be better predicted with the better modeling of muscle activity intensity.

In another embodiment, the user can tag their exercise conditions (e.g., “walking on a grassy surface”, “hiking on a trail”, etc.) manually via a connected device 860 (FIG. 4) such as a Bluetooth-enabled smartphone or through direct gesture to the TENS device (user input 850 in FIG. 4) so that specific activity characteristics can be interpreted with a higher accuracy by the estimator unit 526. In yet another embodiment, contextual tags can also be applied automatically to the activity, e.g., the time of the day, the time since waking up (when sleep monitoring functionality is incorporated into the TENS device), the time before or after a certain amount of activities (e.g., after walking 5000 steps), the user location (e.g., via the indoor/outdoor position system 136 in FIG. 4, which may be a GPS), user skin temperature (e.g., via temperature sensor 137 in FIG. 4), etc.

### Cycling Activity Determination

### Rotational Position Determination

### NEURO-107

Another aspect of the present invention is to automatically determine the rotational position of TENS device 100 on the leg of a user through device position detector unit 528 (FIG. 11). Once TENS device 100 is placed on the leg of a user, it stays in position until it is removed from the body. The placement and removal events can be detected via on-skin detector 265 in the manner previously disclosed.

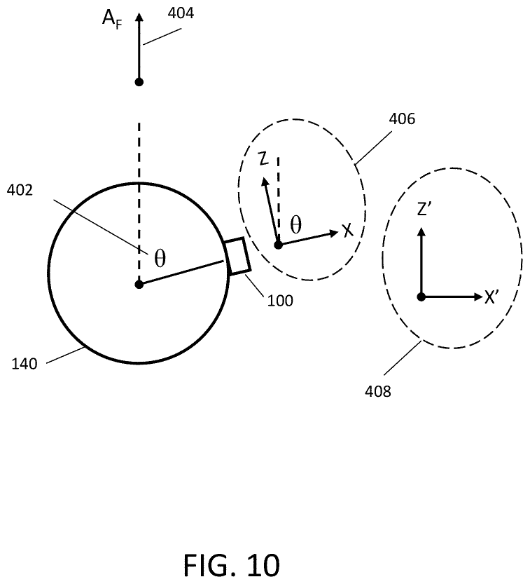

FIG. 10 shows a cross-section (transverse plane) of leg 140 and an exemplary rotational position of TENS device 100 on the leg. The rotational position of TENS device 100 is defined by the angle 402 (denoted as bin FIG. 10) between TENS device 100 and the “forward motion” direction 404 (FIG. 10). It should be noted that the aforementioned stride detection algorithm based on the y-axis accelerometer data from accelerometer 132 functions fully without requiring knowledge of the rotational angle θ.

During the positive phase 334 (FIG. 7) identified by the aforementioned stride detection algorithm, the acceleration associated with forward leg movement (i.e., when the y-axis acceleration value is above the positive zero-crossing threshold 314) is projected onto the x- and z-axis coordinate system 406 (FIG. 10) of accelerometer 132. By way of example but not limitation, if the angle is θ 402 is 90 degrees (i.e., TENS device 100 is placed on the right side of a limb), the forward acceleration A_{F} 404 will have zero projection on the x-axis (A_{F}*cos θ=0) and maximum projection on the z-axis (A_{F}*sin θ=A_{F}). By way of further example but not limitation, if TENS device 100 is placed at the posterior position (i.e., on the back of the leg) with an angle θ=180, the forward acceleration A_{F} 404 will have a negative projection on the x-axis (A_{F}*cos θ=−A_{F}) and a zero projection on z-axis (A_{F}*sin θ=0).

In one preferred embodiment, the x- and z-axis acceleration measurements are acquired during the positive phase 334 (FIG. 7) of leg swing motions. The averages of the x- and z-axis acceleration data over 20 consecutive strides are obtained: these are defined as Āₓ and Ā_{z}. The rotational angle θ 402 is estimated via θ̅=tan ⁻¹(Ā_{z}/Āₓ). Because the periodicity of the tangent function is 180 degrees, the ambiguity of an estimated angle θ̅ belonging to the 0-90 degree range, or belonging to the 180-270 degree range, is resolved based on the signs of Āₓ and Ā_{z}. When the signs of Āₓ and Ā_{z} are both positive, θ̅ belongs in the 0-90 degree range; otherwise θ̅ belongs in the 180-270 degree range.

In one preferred embodiment, an individual estimate of angle θ̅, once it becomes available, is used as the current rotational position of TENS device 100. In another embodiment, the rotational position is a cumulative average of all available individual estimates of the angle obtained since the on-skin event starts. In yet another embodiment, the rotational position of TENS device 100 is a weighted average of the individual angle estimates obtained since the on-skin event starts. In this form of the invention, the angle estimates obtained more recently are given a higher weight factor in the weighted average.

With the knowledge of the rotational position of TENS device 100, the measured accelerations in the coordinate system 406 (FIG. 10) of the x- and z-axis of accelerometer 132 can be mapped to the coordinate system 408 (FIG. 10) of the leg, with an x′-axis considered to be in the medial-lateral direction (i.e., the coronal plane) and the z′-axis considered to be in the anterior-posterior direction (i.e., the sagittal plane) through the well-known “rotation of axes” translation:

A_{x′}=Aₓ sin θ−A_{z} cos θ and A_{z′}=−Aₓ cos θ+A_{z} sin θ.

The mapped values Aₓ, and A_{D} in the x′-z′ axes coordinate system, provide a direct measure of lateral-medial movement (A_{x′}) and anterior-posterior movement (A_{z′}) of the leg and the body. The magnitude and frequency of direction-specific movement allow TENS device 100 to measure other types of activities. In turn, the TENS device can be activated to counter movement-evoked pain as a result of these activities.

One activity often prescribed to fibromyalgia patients is cycling (outdoor or on a stationary bike). With accelerometer data properly mapped to the X′-Y-Z′ coordination system, cycling detector 530 can readily identify cycling exercise activity based on significant periodic movement detected in Y-Z′ plane and little movement in X′ axis. Cycling duration can be measured by tracking the time duration of such periodic movement by the estimator unit 526. The estimator unit 526 can also track cycling activity level by tracking cadence, or pedal revolutions per minute, based on how many cycles of the periodic movement occur in the Y-Z′ plane from the accelerometer data.

It is worth noting that knowledge of the angle θ 402 is not necessary for detecting cycling activity type or measuring the cycling activity duration. Repeated motion of the leg during cycling will always be captured by the accelerometer. Projections of the motion onto accelerometer axes (no matter what the angle θ) will always be periodic but with an unspecific amplitude. Therefore, if one can determine the periodic nature of the leg motion without the impulse-like waveform elements related to heel strike event 339 or toe-off event 338 in walk activity (see FIG. 7), cycling activity type can be inferred and its characteristics can be tracked.

### Other Activity Determination

Strength exercises such as lifting a barbell can also be tracked and monitored by an activity tracker 170 or 172 (FIG. 4) with a generic activity detector 532 (FIG. 11). Movements of the arm can be tracked by an activity tracker attached to the arm. The tracker 170 can be a part of a TENS device if the TENS device 100 is worn on the arm. The tracker 172 can also communicate with the TENS device 100 wirelessly (e.g., via a Bluetooth connection) when the TENS device is placed on another part of the body, such as on the upper calf of a leg. In one embodiment, exercise characteristics (e.g., exercise level and duration) instead of raw accelerometer data are transmitted from the activity tracker to the TENS device. Conversion of the raw accelerometer data to exercise characteristics is done within the tracker with a processing unit connected to the electromechanical sensors. In yet another embodiment, commands to start, to stop, or to modify a TENS therapy, instead of the movement characteristics, are transmitted to the TENS device 100 from an activity tracker 172.

Isometric exercise refers to the physical activity of tensing muscle without any visible body movement and it can be detected by the generic activity detector 532 with appropriate sensor input. An EMG sensor 131 (FIG. 4) can be used to monitor the muscle contractions. An acoustic myograph (AMG) sensor 131 (FIG. 4) can also be used to sense the muscle activity. A stretchable conductive sensor (other sensors 139 in FIG. 4) can also be used to sense the skin stretch due to muscle activities. Similar to the arrangements for strength exercise monitoring, EMG or AMG sensor data, muscle contraction characteristics based on the sensor data, or TENS device control commands based on muscle characteristics are transmitted from an activity tracker 170 to the TENS device 100 worn in the same part of the body as the activity tracker 170. EMG or AMG sensor data, muscle contraction characteristics based on the sensor data, or TENS device control commands based on muscle characteristics are transmitted from an activity tracker 172 to the TENS device 100 worn in a different part of the body. The transmission can be wired or wireless.

Stretch exercise can be monitored based on its body motion component (similar to strength exercise) and muscle contraction component (similar to isometric exercise).

A user may also engage in guided physical activity exercises such as those carried out in a physical therapy clinic or those carried out with a virtual instructor (e.g., Apple Fitness). In addition to tracking activities through the above-mentioned sensors, activities can also be tracked and measured through User Input 850 by a physical therapist or by a connected device 860 with data from the virtual instructor program.

### Controller For Modifying Stimulation Parameters

The results of the activity type, level, and duration assessments (i.e., output of the estimator 526) of the TENS user can be presented to the user or the caregivers of the user via smartphone 860 or similar connected devices. A greater variety of activity types, a higher activity level, and a longer activity duration are important examples of an improved quality of life and health. These improvements can be attributed to a reduction of pain as a result of motion-activated TENS therapy. Changes in these functions are usually gradual and difficult to quantify. When the TENS users are provided with objective and background measurements of these important health metrics, they are more likely to continue with the TENS therapy.

A key feature of the present invention is that the novel TENS device automatically adjusts its stimulation parameters according to the aforementioned activity type, level, and duration (i.e., the output of the estimator 526) through controller unit 452 (FIGS. 4 and 11). The function to map activity type, level, and duration to TENS control commands (e.g., start stimulation, stop stimulation, adjust stimulation intensity) can start with default settings based on a prior knowledge such as those gained through clinical study observations. For example, a TENS therapy will start automatically when 5 minutes of walking activity at an average speed is detected. The TENS therapy will end when the walking activity is absent for at least 3 minutes. When the walking speed is above the average speed of 3.5 miles per hour, the walking activity level is considered high. A high walking activity level will shorten the activity duration required to start a TENS therapy from 5 minutes to 3 minutes. Alternatively, a high walking activity level will automatically increase the TENS stimulation intensity by 20%. A high walking activity level can also lead to both a reduced duration to start a therapy and an increased therapy intensity. For activities other than walking, a mapping function can be similarly established.

The mapping function can be modified based on usage patterns of individual TENS users. For example, if the activity is frequently interrupted by a reduced activity level or a pause of the activity, the interruption may be due to insufficient pain control of the TENS device. The activity duration required to activate TENS therapy may be too long for the TENS user. The function can learn from this pattern by temporarily activating TENS therapy earlier (i.e., with a shorter activity duration threshold). If subsequent user activity level becomes steadier and/or activity duration becomes longer, the shortened activity duration will permanently replace the default duration settings for that user. Similar updates can also be made for stimulation intensity adjustment.

The mapping function default settings for a new TENS user can be modified based on usage patterns of one or more existing TENS users. Adjustments to the default settings as described in the previous paragraph can be captured in a database accessible to all TENS users. When the TENS device of a new user connects to the database, updated duration threshold can be adopted by the TENS device. Adoption of settings in the database can be universal or personalized. Universal adoption means that TENS devices for all new users will receive the same update of the default settings based on the usage patterns of all existing users. Personalized adoption means that TENS devices for a new user will receive an update of the settings based on a subset of the existing users whose profiles match the profile of the new user. Elements of the profile may include age, gender, height, weight, medical history, body temperature, pain conditions (such as pain location), pain patterns (such as pain frequency), electrode-skin impedance, TENS usage pattern (such as body location where the TENS device is placed), activity type, geographic location, and weather condition. Matching can be for all available elements or only selected elements in the profile.

### Exemplary Operation

In one preferred form of the invention, TENS device 100 comprises a stimulator 105 (FIG. 2), an on-skin detector 265 (FIG. 4), a device position detector 528 (FIG. 11), a controller 452 (FIG. 4) for modifying stimulation parameters, and a processor 515 (FIG. 4) for analyzing activity type, activity level, activity duration, and device position. TENS device 100 is preferably configured/programmed to operate in the manner shown in FIGS. 4 and 11, among others.

More particularly, when TENS device 100 is secured to the upper calf 140 of the user, on-skin detector 265 communicates with one or more electromechanical sensors 132 (such as a gyroscope and/or an accelerometer) to indicate that an on-skin session has started and data from the electromechanical sensors 132 are processed to determine the user's activity measurements. The data will also be used to determine the placement position (including the limb) of TENS device 100 on the user.

At the onset of an on-skin session, the orientation of TENS device 100 is set to assume an upright orientation by device orientation detector 512. Based on accelerometer y-axis data, device orientation detector 512 will update the device orientation to either a confirmed upright status or a confirmed upside-down status. The confirmed status (upright or upside-down) will then be persistent until the on-skin session ends. A confirmed upside-down device orientation will cause accelerometer values in x- and y-axis to reverse their signs. With the sign-reversal, the data stream from a gyroscope or an accelerometer can be processed in the same manner for either device orientation status.

Although the y-axis of the accelerometer 132 is approximately along the same direction as gravity when the user is standing, the alignment may not be perfect. As a result, the static gravity projected on the y-axis may not be exactly the same as −1*g. Device vertical alignment unit 514 (FIG. 11) determines the exact alignment relationship between the y-axis and gravity, and alignment results are used to remove static gravity to obtain net activity acceleration for any activity associated with upright body orientation such as walking and cycling. The alignment results can be updated periodically during the on-skin session. In addition to alignment, device vertical alignment unit 514 (FIG. 11) also determines negative zero-crossing threshold 312 (FIG. 7) and positive zero-crossing threshold 314 (FIG. 7) to define a zero-activity region. The zero-activity region may be updated continuously during the on-skin session.

Filter operation 516 (FIG. 11) applies filters to the y-axis data by removing the static gravity component and smoothing out rapid changes near the zero-activity region. Filtered y-axis data are used to determine the user's activity levels and types. Filter operations such as low-pass filters to remove high-frequency noise can also be applied to x-axis and z-axis accelerometer data.

Leg swing is a critical and necessary component in walking and running. Leg activity classifier unit 518 (FIG. 11) identifies components in the acceleration or gyroscope data waveforms characteristic to leg swings. The timing of events like toe-off and heel strike associated with each leg swing is extracted from the waveform features.

Leg swing is also characteristic of cycling. Unlike walking or running, no impulse-like events (corresponding to heel strike or toe-off) will be present in accelerometer data but the periodic nature of the acceleration will be evident. Repetition of the leg swing motion or pedaling cadence will be at a higher frequency that walking cadence.

Stride counter 520 (FIG. 11) counts the number of strides cumulatively within a specific time period (such as 24-hour period) and results are reported to the user either as a display on TENS device 100 or through a connected device 860 (FIG. 4) linked to the TENS device (such as a smartphone connected to the TENS device via Bluetooth).

Walk detector 522 (FIG. 11) determines whether the user is walking by monitoring timing patterns of detected swing events. Regular occurrences of swing events with occurrence intervals between one-half second and 2 seconds are indicative of a walking period. It should be noted that the occurrence interval can be adapted to determine jogging or running. Cycling activity can be detected by a cycling detector unit 530 similarly based on repetition of the leg swing motion for a minimum period of time.

Once walking or cycling activity is detected, the activity duration can be measured through a timer or a real-time clock 135 (FIG. 4). The timer will only stop when the tracked activity is no longer present.

When the activity duration meets the duration threshold (e.g., 10 minutes) to start a TENS therapy, the TENS device will automatically start a TENS therapy. In one preferred embodiment, the TENS therapy will last for a pre-determined time period (e.g., 60 minutes). In another embodiment, the TENS therapy will end after the monitored activity has stopped for a period of time (e.g., 15 minutes). In yet another embodiment, the TENS therapy will end at the later time of the previously-stated events (i.e., after a fixed time period or the termination of the monitored activity type).

Gait analyzer 524 (FIG. 11) receives input from leg activity classifier 518 (stride duration defined as time difference between consecutive toe-off events), stride counter 520 (the number of strides in a walk segment), and walk detector 522 (walking status) to determine whether a sufficient number of strides have been accumulated to perform gait variability analysis. If enough stride durations are collected and the stride duration sequence has a sufficient length without outliers, stride variability measures are calculated for the walk segment by gait analyzer 524. One such measure is the coefficient of variation (CoV), defined as the standard deviation divided by the mean of the stride duration sequence (expressed as a percentage value). If the walk segment duration exceeds the duration threshold above which the TENS therapy is scheduled to start, the CoV value can also be calculated based on the walk segment for that initial duration. The CoV value itself, or the value normalized by the historical values (such as minimum, median, or maximum as discussed below) can be used to estimate the activity level as discussed earlier. The CoV can be updated continuously so that the walking activity level can be monitored and used in real time to adjust the TENS stimulation intensity.

Like gait analysis, cycling cadence can be tracked over time through cycling detector 530 to determine the activity level. If the cadences are high (i.e., the CoV of successive periods of leg swing motion during cycling activity is high), then the activity level is considered high. Interpretation of the CoV can be based on a universal threshold value or historical values collected for the specific individual.

Device position detector 528 (FIG. 11) determines the rotational position of TENS device 100 on leg 140. During a swing phase, detector 528 estimates the forward motion acceleration vector direction in the plane defined by the x- and z-axis of accelerometer 132 based on the x- and z-axis data. The rotational angle θ 402 (FIG. 10) is estimated based on the projection of the acceleration vector A_{F} 404 (FIG. 10) onto the x- and z-axes. The rotational position angle θ 402 can be continuously refined as more measurement data became available. The total duration of the same device position across multiple on-skin sessions within a set period of time (such as a 24-hour day) can be used to inform the user to prevent skin irritation. This is because it is generally advisable to air-out the skin under the TENS device from time to time to minimize the risk of skin irritation. Device position can also be used to control stimulation parameters as the nerve sensitivity at different locations of upper calf may be different.

Other activities such as strength, stretch, and isometric exercise can be monitored by the generic activity detector 532, and their activity duration and level can be similarly quantified. When EMG or AMG sensor 131 detects muscle contractions and accelerometer 132 detects very little physical activity, activity type is registered as isometric exercise. Data from the stretchable conductive sensor (as a part of other sensor 139) can also be optionally used to refine the detection results of the activity detector 532. Short-term energy in the EMG or AMG signal can be used to quantify the activity level. One such implementation of short-term energy is to add the squared signal amplitude over a specific period (e.g., every five seconds). Body movements that lack consistency in motion repetition are registered as stretch or strength exercise. To register strength and stretch activities, more than one activity tracker may be placed on the user's body. The TENS device may be co-located with one of the trackers. The TENS device may also be placed in a body location that is different from all sensor locations.

### Modifications Of The Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. Apparatus for providing transcutaneous electrical nerve stimulation (TENS) therapy to a user, said apparatus comprising:
a stimulation unit for electrically stimulating at least one nerve of the user;
a sensing unit for sensing body movement of the user to analyze body movement activity type and activity duration;
an application unit for providing mechanical coupling between said sensing unit and the user's body; and
a feedback unit for at least one of (i) providing the user with feedback in response to said analysis of said body movement activity type and activity duration of the user, and (ii) modifying the electrical stimulation provided to the user by said stimulation unit in response to said analysis of said body movement activity type and activity duration of the user.

2. Apparatus according to claim 1 wherein said sensing unit uses data from an electromechanical sensor.

3. Apparatus according to claim 2 wherein said electromechanical sensor comprises at least one of (i) an accelerometer, and (ii) a gyroscope.

4. Apparatus according to claim 1 wherein said sensing unit uses data from an electrophysiological sensor.

5. Apparatus according to claim 4 wherein said electrophysiological sensor comprises at least one of (i) an electromyography sensor, and (ii) an acoustic myography sensor.

6. Apparatus according to claim 1 wherein said sensing unit uses data from a force sensitive sensor.

7. Apparatus according to claim 1 wherein said sensing unit uses data from a stretchable conductive sensor.

8. Apparatus according to claim 1 wherein said application unit is a flexible band.

9. Apparatus according to claim 1 wherein said application unit determines whether said sensing unit is mechanically coupled to the body of the user.

10. Apparatus according to claim 9 wherein said application unit uses an on-skin detector to determine mechanical coupling between said sensing unit and the body of the user.

11. Apparatus according to claim 9 wherein said application unit uses a tension gauge to determine mechanical coupling between said sensing unit and the body of the user.

12. Apparatus according to claim 9 wherein the determination of whether said sensing unit is mechanically coupled to the body of the user determines the usability of the data from said sensing unit.

13. Apparatus according to claim 1 wherein said body movement is detectable physical movement of the body of the user.

14. Apparatus according to claim 1 wherein said body movement is muscle movement of the user.

15. Apparatus according to claim 1 wherein said body movement activity type is walking.

16. Apparatus according to claim 1 wherein said body movement activity type is cycling.

17. Apparatus according to claim 1 wherein said body movement activity type is stretch exercise.

18. Apparatus according to claim 1 wherein said body movement activity type is strength exercise.

19. Apparatus according to claim 1 wherein said body movement activity type is guided physical activity.

20. Apparatus according to claim 15 wherein said body movement activity type is determined to be walking when a processed feature of data from said sensing unit is determined to be stepping continuously for a period of time.

21. Apparatus according to claim 20 wherein said period of time is 20 seconds.

22. Apparatus according to claim 1 wherein said activity duration is the time period during which said body movement activity type persists.

23. Apparatus according to claim 1 wherein said sensing unit analyzes body movement activity level of the user.

24. Apparatus according to claim 1 wherein said feedback unit is activated when the said activity duration exceeds an activity duration threshold corresponding to said body movement activity type.

25. Apparatus according to claim 24 wherein said activity duration threshold is a fixed value.

26. Apparatus according to claim 25 wherein said fixed value is 5 minutes.

27. Apparatus according to claim 24 wherein said activity duration threshold is a function of at least one of (i) said body movement activity type, (ii) a body movement activity level, (iii) demographic information of the user, (iv) clinical characteristics of the user, and (v) usage information of other users.

28. Apparatus according to claim 1 wherein said feedback unit provides feedback to the user via an alert delivered to the user through at least one of (i) a smartphone, and (ii) another connected device.

29. Apparatus according to claim 1 wherein said feedback unit provides feedback to the user in the form of mechanical vibrations provided to the user.

30. Apparatus according to claim 1 wherein said feedback unit provides feedback to the user in the form of electrical stimulation provided to the user.

31. Apparatus according to claim 1 wherein said feedback unit modifies said electrical stimulation when said activity duration exceeds an activity duration threshold corresponding to the body movement activity type.

32. Apparatus according to claim 1 wherein said electrical stimulation modification is to change stimulation intensity.

33. Apparatus according to claim 1 wherein said electrical stimulation modification is to change stimulation frequency.

34. Apparatus according to claim 1 wherein said electrical stimulation modification is to change Stimulation start time.

35. Apparatus according to claim 1 wherein said electrical stimulation modification is to change stimulation stop time.

36. Apparatus according to claim 1 wherein said electrical stimulation modification is to change stimulation duration.

37. Apparatus according to claim 1 wherein said electrical stimulation modification is to change stimulation pulse patterns.

38. A method for applying transcutaneous electrical nerve stimulation to a user, said method comprising the steps of:
applying a stimulation unit and a sensing unit to the body of the user;
using said stimulation unit to deliver electrical stimulation to the user so as to stimulate one or more nerves of the user;
analyzing data collected by said sensing unit to determine the user's body movement activity type and activity duration; and
modifying the electrical stimulation delivered by said stimulation unit based on the analysis of body movement activity type and activity duration.
